SEQUENCE LISTING (22 sequences)

SEQ ID NO: 1 - protein, 9 aa, Homo sapiens
ISSCGFYWN

SEQ ID NO: 2 - protein, 12 aa, Homo sapiens
IGYIYYSGSTYY

SEQ ID NO: 3 - protein, 9 aa, Homo sapiens
STRLRGADY

SEQ ID NO: 4 - protein, 17 aa, Homo sapiens
KSSQSVLYNSNNKKYLA

SEQ ID NO: 5 - protein, 7 aa, Homo sapiens
WASTRES

SEQ ID NO: 6 - protein, 9 aa, Homo sapiens
QQYYSTPWT

SEQ ID NO: 7 - protein, 119 aa, Homo sapiens
QVQLQESGPGLVKPSQTLSLTCTVSGGSISSCGFYWNWIRQHPGKGLEWIGYIYYSGSTYYNPSLKSRVTISLDTSKSQFSLKLSSLTAADTAVYYCARSTRLRGADYWGQGTMVTVSS

SEQ ID NO: 8 - protein, 114 aa, Homo sapiens
DIVMTQSPDSLAVSLGERATINCKSSQSVLYNSNNKKYLAWYQQKPGQPPKLLIYWASTRESGVPDRFSGSGSGTDFTLTISSLQAEDVAVYYCQQYYSTPWTFGQGTKVEIKR

SEQ ID NO: 9 - protein, 8 aa, Homo sapiens
LVWVPSDK

SEQ ID NO: 10 - protein, 16 aa, Homo sapiens
VSHLLGINVTDFTRGI

SEQ ID NO: 11 - protein, 12 aa, Homo sapiens
EQADFAIEALAK

SEQ ID NO: 12 - protein, 17 aa, Homo Sapiens
DVDRIIGLDQVAGMSET

SEQ ID NO: 13 - protein, 15 aa, Homo Sapiens
TELEDTLDSTAAQQE

SEQ ID NO: 14 - protein, 12 aa, Homo Sapiens
ALESQLQDTQEL

SEQ ID NO: 15 - protein, 16 aa, Homo Sapiens
SMEAEMIQLQEELAAA

SEQ ID NO: 16 - protein, 17 aa, Homo Sapiens
RRLEARIAQLEEELEEE

SEQ ID NO: 17 - protein, 1960 aa, Homo Sapiens
MAQQAADKYLYVDKNFINNPLAQADWAAKKLVWVPSDKSGFEPASLKEEVGEEAIVELVENGKKVKVNKDDIQKMNPPKFSKVEDMAELTCLNEASVLHNLKERYYSGLIYTYSGLFCVVINPYKNLPIYSEEIVEMYKGKKRHEMPPHIYAITDTAYRSMMQDREDQSILCTGESGAGKTENTKKVIQYLAYVASSHKSKKDQGELERQLLQANPILEAFGNAKTVKNDNSSRFGKFIRINFDVNGYIVGANIETYLLEKSRAIRQAKEERTFHIFYYLLSGAGEHLKTDLLLEPYNKYRFLSNGHVTIPGQQDKDMFQETMEAMRIMGIPEEEQMGLLRVISGVLQLGNIVFKKERNTDQASMPDNTAAQKVSHLLGINVTDFTRGILTPRIKVGRDYVQKAQTKEQADFAIEALAKATYERMFRWLVLRINKALDKTKRQGASFIGILDIAGFEIFDLNSFEQLCINYTNEKLQQLFNHTMFILEQEEYQREGIEWNFIDFGLDLQPCIDLIEKPAGPPGILALLDEECWFPKATDKSFVEKVMQEQGTHPKFQKPKQLKDKADFCIIHYAGKVDYKADEWLMKNMDPLNDNIATLLHQSSDKFVSELWKDVDRIIGLDQVAGMSETALPGAFKTRKGMFRTVGQLYKEQLAKLMATLRNTNPNFVRCIIPNHEKKAGKLDPHLVLDQLRCNGVLEGIRICRQGFPNRVVFQEFRQRYEILTPNSIPKGFMDGKQACVLMIKALELDSNLYRIGQSKVFFRAGVLAHLEEERDLKITDVIIGFQACCRGYLARKAFAKRQQQLTAMKVLQRNCAAYLKLRNWQWWRLFTKVKPLLQVSRQEEEMMAKEEELVKVREKQLAAENRLTEMETLQSQLMAEKLQLQEQLQAETELCAEAEELRARLTAKKQELEEICHDLEARVEEEEERCQHLQAEKKKMQQNIQELEEQLEEEESARQKLQLEKVTTEAKLKKLEEEQIILEDQNCKLAKEKKLLEDRIAEFTTNLTEEEEKSKSLAKLKNKHEAMITDLEERLRREEKQRQELEKTRRKLEGDSTDLSDQIAELQAQIAELKMQLAKKEEELQAALARVEEEAAQKNMALKKIRELESQISELQEDLESERASRNKAEKQKRDLGEELEALKTELEDTLDSTAAQQELRSKREQEVNILKKTLEEEAKTHEAQIQEMRQKHSQAVEELAEQLEQTKRVKANLEKAKQTLENERGELANEVKVLLQGKGDSEHKRKKVEAQLQELQVKFNEGERVRTELADKVTKLQVELDNVTGLLSQSDSKSSKLTKDFSALESQLQDTQELLQEENRQKLSLSTKLKQVEDEKNSFREQLEEEEEAKHNLEKQIATLHAQVADMKKKMEDSVGCLETAEEVKRKLQKDLEGLSQRHEEKVAAYDKLEKTKTRLQQELDDLLVDLDHQRQSACNLEKKQKKFDQLLAEEKTISAKYAEERDRAEAEAREKETKALSLARALEEAMEQKAELERLNKQFRTEMEDLMSSKDDVGKSVHELEKSKRALEQQVEEMKTQLEELEDELQATEDAKLRLEVNLQAMKAQFERDLQGRDEQSEEKKKQLVRQVREMEAELEDERKQRSMAVAARKKLEMDLKDLEAHIDSANKNRDEAIKQLRKLQAQMKDCMRELDDTRASREEILAQAKENEKKLKSMEAEMIQLQEELAAAERAKRQAQQERDELADEIANSSGKGALALEEKRRLEARIAQLEEELEEEQGNTELINDRLKKANLQIDQINTDLNLERSHAQKNENARQQLERQNKELKVKLQEMEGTVKSKYKASITALEAKIAQLEEQLDNETKERQAACKQVRRTEKKLKDVLLQVDDERRNAEQYKDQADKASTRLKQLKRQLEEAEEEAQRANASRRKLQRELEDATETADAMNREVSSLKNKLRRGDLPFVVPRRMARKGAGDGSDEEVDGKADGAEAKPAE

SEQ ID NO: 18 - DNA, 26 bp, Homo Sapiens
cgggtaccatggcacagcaagctgcc

SEQ ID NO: 19 - DNA, 26 bp, Homo Sapiens
gactagtcttctcgtcctccacctgc

SEQ ID NO: 20 - protein, 10 aa, Homo sapiens
ALPGAFKTRK

SEQ ID NO: 21 - protein, 10 aa, Homo sapiens
EELVKVREKQ

SEQ ID NO: 22 - protein, 10 aa, Homo sapiens
ADGAEAKPAE

(19) United States
(12) Patent Application Publication
(10) Pub. No.: US 2010/0226974 A1
(43) Pub. Date: Sep. 9, 2010

(54) ANTIBODY RECOGNIZING ANTIGEN

(75) Inventors: Yoko HIRAKAWA, Tokyo (JP); Hisae NIKI, Tokyo (JP); Shinsuke OIKE, Tokyo (JP); Toshiaki TAGAWA, Tokyo (JP); Saiko HOSOKAWA, Tokyo (JP); Yoshiko YOSHIYAMA, Yokohama-shi (JP)

Correspondence Address: LEYDIG VOIT & MAYER, LTD, TWO PRUDENTIAL PLAZA, SUITE 4900, 180 NORTH STETSON AVENUE, CHICAGO, IL, 60601-6731, US

(73) Assignee: MITSUBISHI TANABE PHARMA CORPORATION, OSAKA-SHI (JP)

(21) Appl. No.: 12/782,834

(22) Filed: May 19, 2010

Related U.S. Application Data
(62) Division of application No. 10/530,171, filed on May 17, 2005, now abandoned.

(30) Foreign Application Priority Data
Oct. 4, 2002 (JP) 2002-291953

(51) Int. Cl. A61K 9/127; A61K 39/395; C07K 16/30

(52) U.S. Cl. 424/450; 424/174.1; 530/391.1

(57) ABSTRACT

The invention provides antibodies against a non-muscular myosin heavy chain type A or a mutant thereof, and F(ab′)₂ fragments thereof, as well as a method of treating cancer with such antibodies and F(ab′)₂ fragments thereof.

### CROSS-REFERENCE TO RELATED APPLICATIONS

This patent application is a divisional of copending U.S. patent application Ser. No. 10/530,171, filed on May 17, 2005, which is the U.S. national phase of International Patent Application No. PCT/JP2003/012732, filed on Oct. 3, 2003, which claims priority to Japanese Patent Application No. 2002-291953, filed on Oct. 4, 2002.

### INCORPORATION-BY-REFERENCE OF MATERIAL ELECTRONICALLY SUBMITTED

Incorporated by reference in its entirety herein is a computer-readable nucleotide/amino acid sequence listing submitted concurrently herewith and identified as follows: 22,685 bytes ASCII (Text) file named “706403SequenceListing.txt,” created May 17, 2010.

### TECHNICAL FIELD

The present invention relates to an antigen having a part which is exposed on the surface of a cell at the formation of a tumor mass. More specifically, the present invention relates to a useful medicament which recognizes, as an antigen, a part of a non-muscular myosin heavy chain type A or a mutant thereof which is exposed to the cell surface in a solid tumor.

### BACKGROUND OF THE INVENTION

Currently, studies on cancer targeting agents using antibodies for cancer cells are in progress to obtain high efficacy and safety as antitumor agents. For example, a human monoclonal antibody screened by its reactivity with gastric cancer and colon cancer is known as GAH antibody (cf. EP-A-526700 and EP-A-520499), and development of a drug-containing liposome on which the antibody was bound (cf. EP-A-526700) is in progress.

On the other hand, it is known that the variation of antibodies which recognize the cells depends on the kinds of cells which proceed to malignancies. When a drug-containing liposome bound to the antibody is used as a cancer targeting agent, it is considered that identification of an antigen which is recognized by the antibody is necessary for obtaining higher efficacy and safety as an antitumor agent. However, regarding the GAH antibody, an antigen recognized by the antibody has not so far been identified.

Also, although there are reports stating that an antibody obtained by immunizing a rabbit with a myosin heavy chain purified from a mouse fibroblast cell line L929 reacts with surfaces of L929 and other cells [cf. Willingham M. C., Proc. Natl. Acad. Sci. U.S.A., 71, 4144 (1974), and Olden K., Cell, 8, 383-390 (1976)], there are no reports on the exposure of a human non-muscular myosin heavy chain type A (hereinafter referred sometimes to as “nmMHCA”) to the cell surface by carcinogenesis, and there are no reports in which the protein is a cancer-associated antigen.

### Patent Reference 1: EP-A-526700

### Patent Reference 2: EP-A-520499

Non-patent Reference 1: Willingham M. C., Proc. Natl. Acad. Sci. U.S.A., 71, 4144 (1974)

### Non-patent Reference 2: Olden K., Cell, 8, 383-390 (1976)

### BRIEF SUMMARY OF THE INVENTION

In order to solve the above problems, the present inventors have conducted intensive studies and found as a result that an antibody represented by the human monoclonal antibody disclosed in EP-A-520499 (GAH antibody) recognizes an antigen having a part which is exposed on the surface of a cell at the formation of a tumor mass in the cell.

That is, the present invention relates to the followings.

(1) An antigen having a part which is exposed on the surface of a cell at the formation of a tumor mass.
(2) The above antigen, wherein the tumor mass is a solid tumor formed by subcutaneous transplantation of a cultured cancer cell.
(3) The above antigen, wherein the existing amount of the antigen of the solid tumor is increased in comparison with that of a cultured cell of the solid tumor.
(4) The above antigen, wherein the existing amount of the antigen of the solid tumor on the cell surface is increased in comparison with that of a cultured cell of the solid tumor.
(5) The above antigen, which is a cytoskeleton protein or a mutant thereof.
(6) The above antigen, which is myosin or a mutant thereof.
(7) The above antigen, which is a non-muscular myosin heavy chain type A or a mutant thereof.
(8) The above antigen, which is a part of a non-muscular myosin heavy chain type A or a mutant thereof.
(9) The above antigen, which is a sequence of a C-terminal domain of the protein sequence of a non-muscular myosin heavy chain type A or a mutant thereof.
(10) The above antigen, wherein the sequence of a C-terminal domain of the protein sequence is a sequence of the residue at position 600 to the residue at position 1,960 from the N-terminal of SEQ ID NO:17 in the Sequence Listing.
(11) The above antigen, wherein the sequence of a C-terminal domain of the protein sequence is any one of SEQ ID NOs:20, 21 and 22.
(12) A ligand which recognizes the above antigen.
(13) The above ligand, which is an antibody.
(14) The above ligand, which is a monoclonal antibody.
(15) The above ligand, wherein the monoclonal antibody is a human monoclonal antibody.
(16) The above ligand, which is a cancer reactive monoclonal antibody.
(17) The above ligand, wherein the cancer is gastric cancer, breast cancer, colon cancer or esophageal cancer.
(18) The above ligand, wherein the heavy chain hypervariable region comprises the amino acid sequences of SEQ ID NOs:1, 2 and 3 in the Sequence Listing, and the light chain hypervariable region comprises the amino acid sequences of SEQ ID NOs:4, 5 and 6 in the Sequence Listing.
(19) The above ligand, which comprises a heavy chain variable region comprising the amino acid sequence of SEQ ID NO:7 in the Sequence Listing and a light chain variable region containing the amino acid sequence of SEQ ID NO:8 in the Sequence Listing.
(20) A pharmaceutical composition, which comprises the above ligand.
(21) The above pharmaceutical composition, which is a targeting therapy agent.
(22) The above pharmaceutical composition, which targets at a cancer tissue or a cancer cell.
(23) The above pharmaceutical composition, which comprises an antitumor agent, an antitumor protein, an enzyme, a gene or an isotope for treatment.
(24) The above pharmaceutical composition, which is an antitumor agent.
(25) The above pharmaceutical composition, wherein the cancer is gastric cancer, breast cancer, colon cancer or esophageal cancer.
(26) The above pharmaceutical composition, which comprises liposome.
(27) A labeling agent, which comprises the above ligand.
(28) The above labeling agent, which specifically labels a cancer tissue or a cancer cell.
(29) The above labeling agent, wherein the cancer is gastric cancer, breast cancer, colon cancer or esophageal cancer.
(30) The above labeling agent, which comprises a fluorescent, an enzyme, an isotope or an MRI contrast medium.
(31) A method for treating a cancer disease of a cancer disease patient which expresses the above antigen, which comprises administering the above pharmaceutical composition.
(32) A method for treating a cancer disease of a cancer disease patient having a cell which can be labeled by the above labeling agent, which comprises administering the above pharmaceutical composition.
(33) The above ligand, wherein the binding activity of the ligand which recognizes the above antigen to the antigen is from 0.5×10⁶ units/mg to 2.0×10⁶ units/mg.
(34) The above ligand, wherein the binding activity is from 0.7×10⁶ units/mg to 1.5×10⁶ units/mg, from 0.7×10⁶ units/mg to 1.3×10⁶ units/mg, or from 0.8×10⁶ units/mg to 1.2×10⁶ units/mg.
(35) The above ligand, wherein the binding activity is from 0.8×10⁶ units/mg to 1.2×10⁶ units/mg.

### BRIEF DESCRIPTION OF THE DRAWINGS

FIG. 1 shows reactivity of GAH antibody with cultured cells and transplantation-derived cells of MKN45.

FIG. 2 is a photograph showing results of the evaluation of reactivity of GAH and anti-nmMHCA antibodies in tissue sections of stably expressing nmMHCA.

FIG. 3 shows GAH antibody binding numbers, as a result of the evaluation of cell surface reactivity of GAH using a cell line stably expressing nmMHCA.

FIG. 4 shows anti-nmMHCA antibody binding numbers, as results of the evaluation of cell surface reactivity of anti-nmMHCA antibody using a cell line stably expressing nmMHCA.

FIG. 5 shows results of the evaluation of reactivity of anti-nmMHCA peptide antibody to the surface of MKN45 cultured cells and tumor cells formed by subcutaneous transplantation of MKN45.

FIG. 6 shows a relationship between the tumor growth inhibitory effect and the antigen amount per cell.

### DETAILED DESCRIPTION OF THE INVENTION

The present invention is described below in detail.

The cell of the present invention includes cells derived from stomach, large intestine, esophagus, mammary gland, lungs, pancreas, liver, kidney, ovary or uterus. Preferred examples include cells derived from stomach, large intestine, esophagus or mammary gland. More preferred examples include cells derived from large intestine.

The tumor mass of the present invention may be any tumor mass, so long as it forms an aggregate of tumor cells visibly or microscopically. Preferred examples include those in which a normal tissue is spontaneously transformed to solid tumor by tumorigenesis and those in which a cancer cell is proliferated by the transplantation of the cell. More preferred is a solid tumor formed by subcutaneous transplantation of a cultured cancer cell.

The term exposure as used herein means that an internal substance appears on the surface of cell membrane or that the internal substance is exposed by the peeling of a substance covering the surface, and preferably it means that a part of an antigen appears on the surface of a cell. More preferably, it means that the C-terminal domain of the protein sequence of an antigen appears on the surface of a cell.

Examples of the antigen of the present invention include a protein, a glycoprotein, a protein-lipid complex and mutants thereof, which are considered to function as a cytoskeleton and an organelle in normal cells. Preferred is one which functions as an antigen when a cell forms a tumor mass. More preferred are myosin, actin, tropomyosin, vimentin, cytokeratin and the like or mutants thereof. Most preferred is human non-muscular myosin heavy chain type A (nmMHCA). Herein, nmMHCA is obtained by preparing a gene in accordance with the method of Toothaker L. E. et al. [Blood, 78(7), 1826-1833 (1991)] or the method of Saez C. G. et al. [Proc. Natl. Acad. Sci. USA, 87(3), 1164-8 (1990, February)], and expressing the protein using the gene in accordance with Molecular Cloning, A Laboratory Manual [Second Edition, Cold Spring Harbor Laboratory Press (1989)].

Examples of the sequence of a C-terminal domain of the protein of the present invention include a sequence of the residue at position 600 to the residue at position 1,960 from the N-terminal side of nmMHCA represented by SEQ ID NO:17 in the Sequence Listing, and preferred examples include the sequence of SEQ ID NO:20, 21 or 22 in the Sequence Listing.

Examples of comparison of the solid tumor of the present invention with a cultured cell of the solid tumor include a method in which cultured cancer cells are compared with solid tumor-derived cancer cells separated from a solid tumor formed by once transplanting the cultured cells to an animal, a method in which cancer cells separated from solid cancer tissues of a patient are compared with cultured cancer cells adapted by once culturing the cancer cells in vitro, and the like, although not limited thereto.

The existing amount on the cell surface of the present invention means the existing amount of an antigen in the entire cell or the existing amount of the antigen only on the cell surface, and preferably means the existing amount of the antigen only on the cell surface. The existing amount on the cell surface can be determined, for example, by flow cytometry, although not limited thereto.

Examples of the increase of the present invention include increase by 3 times or more, preferred examples include increase by 4 times or more, and more preferred examples include increase by 10 times or more.

Examples of the mutant of the present invention include those which have amino acid sequences in which one or several amino acids are deleted, substituted or added, or those in which three-dimensional structure of the normal protein is modified.

Examples of the ligand of the present invention include proteins, for example, various antibodies, growth factors or proliferation factors such as fibroblast growth factor (FGF) and epidermal growth factor (EGF), and preferred examples include antibodies. Also, examples of the antibodies include polyclonal antibodies of various animals, mouse monoclonal antibodies, human-mouse chimeric antibodies or human type monoclonal antibodies and human monoclonal antibodies, and preferred examples include human monoclonal antibodies. More preferred examples include cancer reactive human monoclonal antibodies, and a human monoclonal antibody disclosed in EP-A-520499 (GAH antibody).

In the GAH antibody, the amino acid sequences of SEQ ID NOs:1, 2 and 3 in the Sequence Listing are called hypervariable region among heavy chain variable regions, and the amino acid sequences of SEQ ID NOs:4, 5 and 6 in the Sequence Listing are called hypervariable region among light chain variable regions. The regions determine specificity of immunoglobulin as an antibody and binding affinity of an antigenic determinant with the antibody, and are also called complementarity determining regions. Accordingly, regions other than the hypervariable regions may be derived from other antibodies. That is, it is considered that an antibody having hypervariable regions similar to those of the GAH antibody can be used in the present invention in the same manner as the GAH antibody.

Thus, in a preferred monoclonal antibody to be used in the present invention, the amino acid sequences of SEQ ID NOs:1, 2 and 3 in the Sequence Listing are contained in the heavy chain hypervariable region, and the amino acid sequences of SEQ ID NOs:4, 5 and 6 in the Sequence Listing are contained in the light chain hypervariable region. In these amino acid sequences, generally, SEQ ID NOs:1, 2 and 3 in the Sequence Listing and SEQ ID NOs:4, 5 and 6 in the Sequence Listing are contained in this order from the N-terminal region, in the three hypervariable regions of each chain of the heavy chain and light chain. According to the present invention, those in which modifications such as substitution, insertion, deletion or addition of some amino acids were carried out within a range of not spoiling their reactivity with cancers are also included in the monoclonal antibodies which can be used in the present invention.

The monoclonal antibody of the present invention can be obtained by preparing a hybridoma of a patient-derived lymphocyte with a mouse myeloma cell, and selecting one having the above specified amino acid sequence.

The hybridoma is obtained in accordance with the method of A. Imam et al. [Cancer Research, 45, 263 (1985)], by firstly isolating lymphocyte from a lymph node belonging to a cancer excised from a cancer patient, and fusing it with a mouse myeloma cell using polyethylene glycol. Using supernatants of the thus obtained hybridomas, hybridomas capable of producing antibodies which are positive by enzyme immunoassay for various cancer cell lines fixed with p-formaldehyde are selected and their cloning is carried out.

Subsequently, monoclonal antibodies are purified from supernatants of the hybridomas by a conventional method [R. C. Duhamel et al., J. Immunol. Methods, 31, 211 (1979)] and labeled with a fluorescent material to check their reactivity with intact cancer cell lines, various erythrocytes, leukocytes and so on by flow cytometry. Antibodies which show reactivity with intact cancer cell lines and antibodies which do not show reactivity with erythrocytes and leukocytes are selected. In addition, by comparing the reactivity with cancer cells isolated from a cancer tissue excised from a cancer patient and normal cells isolated from a non-cancer part of the same tissue of the same patient, an antibody in which larger numbers of its molecules are bound to the cancer cells and which does not react with normal cells or has a similar degree of reactivity with an antibody derived from a healthy person.

The nucleotide sequence of DNA encoding the antibody produced by the thus selected hybridoma is obtained, for example, by the following method. From the antibody producing hybridoma, mRNA is prepared by the guanidine thiocyanate-lithium chloride method [Casara et al., DNA, 2, 329 (1983)], and its cDNA library is prepared using oligo(dT) primer. Next, (dG) tailing of cDNA is carried out, and cDNA encoding the antibody is amplified by PCR using a partial sequence as the probe, which is common to poly C which hybridizes with this dG tail and the human antibody heavy chain gene and light chain gene already obtained. Thereafter, the termini of the DNA are smooth-ended, the DNA extracted from a gel by electrophoresis is inserted into a cloning vector such as pUC119, and its nucleotide sequence is determined by the dideoxy method of Sanger et al. [Proc. Natl. Acad. Sci. USA., 74, 5463 (1977)]. Based on this nucleotide sequence, an antibody having the above specified amino acid sequence can be selected.

In addition, the monoclonal antibody to be used in the present invention can also be prepared by genetic engineering techniques.

The particularly preferable monoclonal antibody of the present invention is one in which the heavy chain variable region and the light chain variable region are represented by the amino acid sequences of SEQ ID NOs:7 and 8, respectively, in the Sequence Listing. The nucleotide sequences for the constant regions of the heavy chain and the light chain may be those which have the same sequences described, for example, in Nucleic Acids Research, 14, 1779 (1986), The Journal of Biological Chemistry, 257, 1516 (1982) and Cell, 22, 197 (1980).

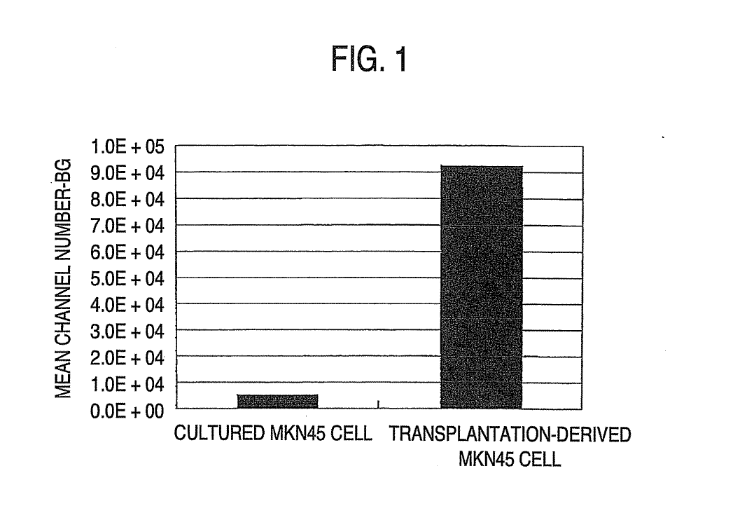

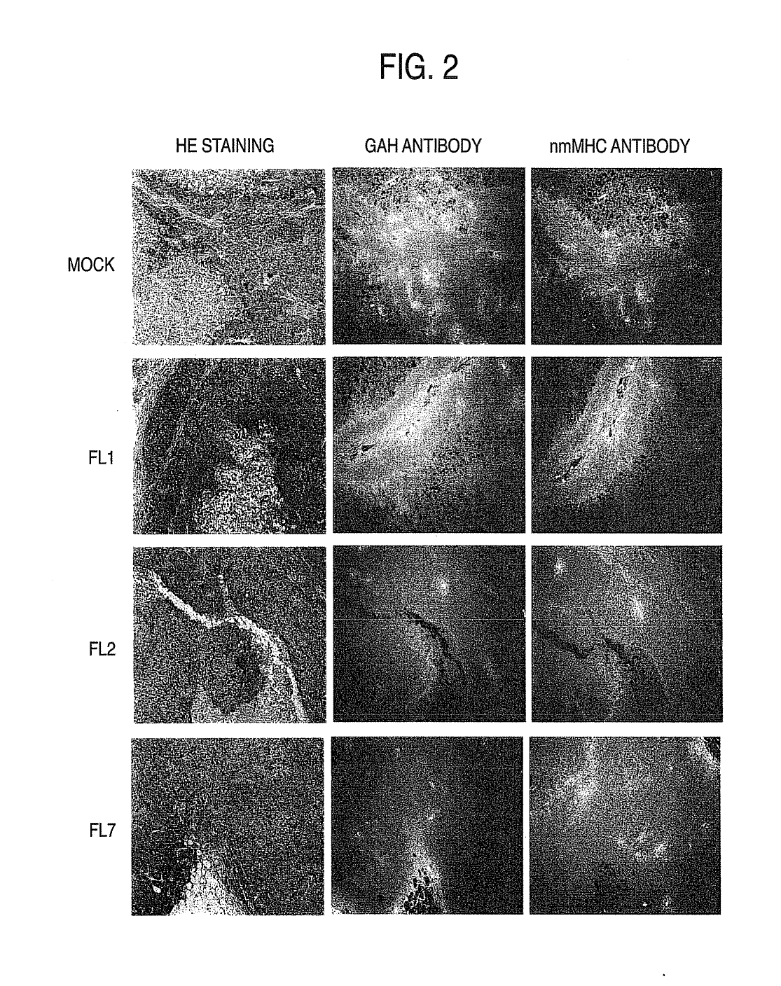

This antibody can be obtained by culturing a hybridoma capable of producing this antibody using fetal bovine serum-containing eRDF, RPMI 1640 culture medium or the like, or by chemically synthesizing a gene in which the DNA molecules encoding variable regions including the above specified hypervariable regions are further ligated with each of DNA molecules encoding constant regions of the heavy chain and the light chain, inserting it into various conventionally known expression vectors capable of expressing the gene, for example, pKCR(ΔE)/H and pKCRD as expression vectors for animal cells, that can be constructed from pKCRH2 [Mishina et al., Nature, 307, 605 (1984)] by the procedure shown in FIG. 1 or FIG. 2 of EP-A-520499, and then expressing it in a host such as CHO cell (Chinese hamster ovary cell). For example, a HindIII site is added to both termini of the heavy chain gene and the product is inserted into the HindIII site of pKCR(ΔE)/H, and a selectable marker gene such as DHFR gene is inserted into SalI site of this plasmid. On the other hand, an EcoRI site is added to both termini of the light chain gene and the product is inserted into EcoRI of pKCRD, and the DHFR gene is also inserted into SalI site of this plasmid. By introducing both plasmids into a cell such as CHO dhfr⁻ [Urlaub G. & Chasin L. A., Proc. Natl. Acad. Sci. USA., 77, 4216 (1980)] by the calcium phosphate method and culturing the cell using nucleotide-free aMEM culture medium or the like, a cell capable of producing the antibody can be further obtained by selecting from the cells which can grow in the medium. The antibody is purified from a culture medium obtained by culturing these cells by adsorbing it to a column or the like in which protein A is linked to a carrier such as Cellulofine or agarose, and then eluting it.

Examples of the cancer of the present invention include cancers having a possibility in that the antibody has a reactivity therewith when, for example, the antibody is used as a single chain antibody (scFv), a whole antibody or a fragment thereof. Preferred examples include gastric cancer, colon cancer, esophageal cancer, lung cancer, breast cancer, hepatic cancer, ovarian cancer, uterine cancer and pancreatic cancer. More preferred examples include gastric cancer, breast cancer, colon cancer and esophageal cancer.

Examples of the pharmaceutical composition of the present invention include a ligand alone and a targeting therapy agent in which an active substance is linked to the ligand, and preferred examples include the targeting therapy agent. Examples of the targeting therapy agent include those in which an active substance is directly linked to a ligand, an active substance is linked to the ligand via a water-soluble polymer or an active substance-containing fine particle is linked to the ligand.

Examples of the fine particle include microsphere, micelle and liposome, and preferred examples include liposome. A pharmaceutical preparation and a labeling agent may be contained in the liposome. Examples of the pharmaceutical preparation to be contained therein include antitumor agents such as adriamycin, daunomycin, vinblastine, cisplatin, mitomycin, bleomycin, actinomycin and fluorouracil (5-FU), pharmaceutically acceptable salts and derivatives thereof. Further examples include a toxic proteins such as ricin A and diphtheria toxin and DNAs encoding them, DNA encoding the cytokine gene of FNF or its antisense DNA, and nucleotides. Particularly preferred examples include adriamycin. Also, examples of the labeling agent to be contained therein include an imaging agent of radioactive element such as indium or technetium, enzymes such as horseradish peroxidase and alkaline phosphatase, MRI contrast medium of gadolinium, X-ray contrast medium of iodine, ultrasonic contrast medium of CO₂, europium derivatives, fluorescent of carboxyfluorescein or illuminant of an N-methylacridium derivative. Examples of the water-soluble polymer derivative include a synthetic polymer of polyethylene glycol, polyacrylamide, polyvinyl pyrrolidone, polyglycerol, polylactic acid, polyglycolic acid or polyamino acid, and preferred examples include polyethylene glycol. It is preferred that the targeting therapy agent targets at a cancer tissue or a cancer cell. Examples of the cancer in this case include gastric cancer, colon cancer, esophageal cancer, lung cancer, breast cancer, hepatic cancer, ovarian cancer, uterine cancer and pancreatic cancer. More preferred examples include gastric cancer, breast cancer, colon cancer and esophageal cancer.

According to the present invention, the activity of an antigen-recognizing ligand to bind to the antigen is measured for, for example, ensuring constancy of the quality of a ligand when the ligand capable of recognizing the antigen is industrially used, though not limited to this purpose. In addition, as the measuring method, for example, there is a method in which a calibration curve is prepared from standard solutions containing predetermined amounts of the antibody using a microplate reader or the like, and the antigen-binding activity of the antibody contained in the test solution is measured, although not limited to this measuring method.

When the binding activity is expressed by a titer, a range of from 0.5×10⁶ to 2.0×10⁶ units/mg can be exemplified, and it is preferably within the range of from 0.7×10⁶ to 1.5×10⁶ units/mg, from 0.7×10⁶ to 1.3×10⁶ units/mg or from 0.8×10⁶ to 1.2×10⁶ units/mg. It is more preferably from 0.8×10⁶ to 1.2×10⁶ units/mg.

The complex of a ligand-linked active substance and a water-soluble polymer derivative can be made into a pharmaceutical preparation, for example, by the method described in EP-A-526700 or EP-A-520499, and the complex can be administered to patients by intravascular administration, bladder administration, intraperitoneal administration, topical administration or the like for the treatment of various diseases such as a cancer. The dose can be optionally selected according to the kind of the antitumor substance as the active ingredient, and when liposome containing doxorubicin is administered, for example, it can be used in 50 mg/kg or less, preferably 10 mg/kg or less, and more preferably 5 mg/kg or less, as the amount of the active ingredient.

### EXAMPLES

The present invention is described below in more detail with reference to examples, but the present invention is not restricted by the following examples without overstepping its gist.

### Example 1

### Reactivity Comparison of Antibodies and Detection of Surface Antigen by Immunoprecipitation

### Preparation of Tumor Cell by Subcutaneous Transplantation of MKN45

A human gastric cancer cell MKN45 (Immuno-Biological Laboratories) was cultured in a liquid medium eRDF (Gibco) containing 10% fetal bovine serum (Sigma), and the resulting cells were recovered and subcutaneously transplanted under the back of an about 5-week-old BALB/C node mouse (CLEA Japan). The thus formed subcutaneous tumor tissue was extracted, and a cell was isolated from the tissue in accordance with the method of Tokita et al., [Gan no Rinsho (Clinical Cancera), 32, 1803 (1986)]. The tissue was put on a Teflon sheet spread on a rubber plate, cut into thin pieces by tapping with a razor, and then passed through a nylon mesh (a cell strainer, FALCON) to remove the connective tissue. Cell suspension as the filtrate was centrifuged at 1,500 rpm for 5 minutes (Tomy table centrifuge LC06-SP), and the floated fat and suspended necrosis moiety were discarded and the precipitate was repeatedly washed.

### Reactivity Comparison of GAH Antibody by Flow Cytometry

Cultured cells of MKN45 or cells of a solid tumor (hereinafter referred to as “tumor cells”) formed by subcutaneous transplantation of MKN45 were allowed to react with the GAH antibody described in EP-A-520499 and EP-A-1174126, which had been labeled with fluorescein isothiocyanate (FITC: Sigma), in a concentration of 20 μg/ml at 4° C. for 1 hour, and then washed once with phosphate buffered saline (PBS). Analysis was carried out in PBS containing propidium iodide (PI: Sigma) using a flow cytometer (FACScan: Becton Dickinson). PI-positive cells, namely dead cells, were excluded from the subject of analysis by gating operation. By calculating the mean channel number of FL1 as an index of FITC fluorescence intensity, comparison of the cultured cells and tumor cells of MKN45 was carried out.

The results are shown in FIG. 1. The ordinate shows a value obtained by subtracting a value in the case of not containing the antibody as a background value (BG) from the mean channel number. E is the power of 10.

The GAH antibody showed a higher reactivity of about 18 times for the tumor cells than the cultured cells of MKN45.

Based on this result, it was found that the GAH antibody shows a higher reactivity for the tumor cells in comparison with the cultured cells.

### Biotin Labeling of the Surface of Cells and Preparation of Solubilized Supernatant

To cultured cells or tumor cells of MKN45, 1 mg/ml PBS solution of a biotin reagent (sulfoNHS-biotin: PIERCE) was added, followed by incubation at 4° C. for 30 minutes on a shaker. Thereafter, the cells were washed with PBS containing 5 mM glycine (Nacalai Tesque) and then with PBS. Pellet of the cells was mixed with a solution of 1% NP 40, aprotinin (Sigma) and nafamostat mesylate (Torii Yakuhin) in 20 mM Tris (Sigma) hydrochloride buffer, pH 7.5, containing 150 mM NaCl and 1 mM EDTA (TNE buffer), stirred, subjected to ultrasonic treatment, allowed to stand on ice for 1 hour and then centrifuged (Tomy table cooling centrifuge MRX-150), and the supernatant was used as a solubilized supernatant of cells.

### Immunoprecipitation and Detection of Biotin-Labeled Band

A solution of GAH antibody or a healthy person-derived human immunoglobulin (human Igs) (purified from human serum obtained from Scanty Bodies Laboratory using a protein A column (Repligen)) was added to protein A Sepharose CL4B (Pharmacia) which had been equilibrated with PBS, and the thus antibody-linked resin was washed with PBS, and the solubilized supernatant of cells was added thereto, followed by incubation at 4° C. overnight on a shaker. After discarding the supernatant by centrifugation, the resin was washed 3 times with THE buffer containing 0.1% NP 40 (Nacalai Tesque) and extracted with a sample buffer for SDS polyacrylamide gel electrophoresis (SDS-PAGE), and the extract was subjected to SDS-PAGE (4 to 12% gradient gel) and then to Western blotting on PVDF membrane (Millipore). The protein-transferred membrane was incubated at room temperature for 1 hour in PBS containing 0.1% gelatin (Nacalai Tesque) and 0.05% Tween 20 (Nacalai Tesque), and then, in order to detect the biotin-labeled protein, allowed to react with VECTASTAIN™ Elite ABC reagent (Vector) at room temperature for 1 hour. Konica Immunostain HRP 1000 (Konica) was used for the color development. The sample used in lane 1 is an immunoprecipitation product by the GAH antibody of the tumor cell, the sample used in lane 2 is an immunoprecipitation product by the GAH antibody of cultured cell, the sample used in lane 3 is an immunoprecipitation product by the human Igs of tumor cell, and the sample used in lane 4 is an immunoprecipitation product by the human Igs of cultured cell.

On the immunoprecipitation product by the GAH antibody of the tumor cell of lane 1, a GAH antibody-specific band was detected at a position of about 200 kd in molecular weight. A band was hardly detected at the corresponding position on the immunoprecipitation product by the GAH antibody of cultured cell of lane 2.

It was found from this result that the GAH antibody has a reactivity with a protein of about 200 kd. In addition, it was shown that this protein of about 200 kd is exposed on the tumor cell surface.

### Amino Acid Sequence Analysis of 200 kd Protein

The 200 kd protein specifically detected by immunoprecipitation was cut out from the polyacrylamide gel, treated with lysyl endopeptidase (Wako Pure Chemical Industries) and subjected to reverse phase chromatography, and then amino acid sequence analysis was carried out on the thus obtained peaks (SEQ ID NOs:9 to 16 in the Sequence Listing).

When homology retrieval was carried out based on these sequences, the human non-muscular myosin A chain (nmMHCA) coincided with this 200 kd protein (SEQ ID NO:17 in the Sequence Listing).

From this result, it was found that the protein of 200 kd is nmMHCA.

Detection by Anti-Non-Muscular Myosin Heavy Chain (nmMHC) Antibody

The above immunoprecipitation samples were subjected to SDS-PAGE and Western blotting, incubated at room temperature for 1 hour in PBS containing 0.1% gelatin and 0.05% Tween 20, and then allowed to react at room temperature for 1 hour in a solution of anti-nmMHC rabbit polyclonal antibody (Biomedical Technologies). Normal rabbit immunoglobulin (rabbit IgG: Biogenensis) was used as the negative control of the antibody. After the reaction in a solution of a peroxidase-labeled anti-rabbit IgG (Cappel) as the secondary antibody, color development was carried out by using Konica Immunostain HRP1000. The samples used in lanes 1, 2 and 3 were immunoprecipitation products by the GAH antibody of the tumor cell, and the samples used in lanes 4, 5 and 6 were immunoprecipitation products by the GAH antibody of cultured cell. Lanes 1 and 4 were detected by VECTASTAIN™ Elite ABC reagent, lanes 2 and 5 were detected by ant-nmMHC antibody, and lanes 3 and 6 were detected by normal rabbit IgG.

Bands were detected on the lanes 1, 2 and 5.

Based on this result, it was found that the protein of about 200 kd, namely nmMHCA, is recognized by the GAH antibody in both of the cultured cell and the tumor cell, and nmMHCA is present on the cell surface in the case of the tumor cell.

### Example 2

### Confirmation Using nmMHCA Forced Expression Cell Line

### Preparation of NmMHCA Expression Vector

HA1.0 and HALES (obtained from Robert S. Adelstein) as the nmMHCA gene were digested with a restriction enzyme EcoRI (Takara Bio). On the other hand, a plasmid vector pEF1/Myc-HisB for mammalian cell gene expression (Invitrogen) into which the nmMHCA gene is to be introduced was also digested with the restriction enzyme EcoRI and then dephosphorylated. The nmMHCA gene fragments and pEF1/Myc-HisB fragments were ligated and transformed to obtain a sample into which HA1.0 was introduced (named pEF1B-HA1.0) and a sample into which HALES was introduced (named pEF1B-HALES). A vector for full length nmMHCA mammalian cell expression was prepared by PCR (Advantage cDNA PCR kit, Clontech) using pEF1B-HA1.0 as the template and using the primer of SEQ ID NO:18 in the Sequence Listing and the primer of SEQ ID NO:19 in the Sequence Listing. This PCR product was named KpnI-HA1.0-SpeI. On the other hand, pEF1B-HALES was digested with restriction enzymes KpnI and SpeI (both Takara Bio). The KpnI-HA1.0-SpeI was inserted into the pEF1B-HALES restriction enzyme digest and then transformation of Escherichia coli was carried out. By carrying out mapping of plasmids of the thus obtained clones, it was confirmed that the vector of interest for full length nmMHCA mammalian cell expression was prepared.

### Preparation of COS-7 Forced Expression Cell Line

The nmMHCA gene was introduced into the plasmid vector pEF1/Myc-HisB for mammalian cell gene expression (Invitrogen), and gene transfer into a Cercopithecus aetiopus kidney-derived cultured cell line COS-7 cell (obtained from Cell Resource Center for Biomedical Research, Institute of Development, Aging and Cancer, Tohoku University) was carried out by lipofection using PolyFect (Qiagen). The gene-transferred cells were cultured at 37° C. in the presence of 5% CO₂ and used after 48 hours in the immunoprecipitation test as transient expression cell lines. After the gene transfer, a stable expression cell line was established by culturing at 37° C. in the presence of 5% CO₂, followed by drug selection of a stable expression cell line using Genetecin G418 (Sigma). In addition, for the purpose of taking influences of gene transfer operation and drug selection operation into consideration in carrying out tests using the nmMHCA stable expression cell line, a mock cell of COS-7 was prepared as the negative control. The mock cell was prepared by carrying out the drug selection after transferring the gene into COS-7 cell by lipofection using a plasmid moiety (pEF1/myc-HisB) alone of the plasmid used in integrating the nmMHCA gene.

### Preparation of HCT-15 Stable Expression Cell Line

Preparation of an NmMhca Gene-Introduced Stable Expression Cell Line was carried out in the same manner as the case of COS-7, using a human colon cancer cell line HCT-15 cell (obtained from Cell Resource Center for Biomedical Research, Institute of Development, Aging and Cancer, Tohoku University). The mock cell was prepared also in the case of HCT-15.

### Immunoprecipitation by GAH Antibody

Each of the nmMHCA transient expression cells and un-introduced cells of COS-7 and HCT-15 were recovered by using a scraper, and 0.5 ml of a solubilization buffer was added thereto, followed by ultrasonic treatment (output 2, frequency 50%) for 5 seconds to disrupt the cells. After allowing to stand on ice for 1 hour, they were centrifuged at 15,000 rpm for 10 minutes using a microtube centrifuge to obtain a solubilized supernatant. In order to make protein concentrations contained in the thus obtained supernatants uniform, determination of protein was carried out by using BCA Protein Assay Kit (Pierce) in the same manner as in Example 1. Immunoprecipitation was also carried out in the same manner as in Example 1, and the immunoprecipitation products and solubilized supernatants for comparison were subjected to SDS-PAGE (gel concentration 6%) and then to Western blotting. The membrane was incubated at room temperature for 1 hour in a blocking buffer (PBS containing 0.1% gelatin and 0.05% Tween 20, 0.05% sodium azide (Wako Pure Chemical Industries)), and the anti-nmMHC antibody was diluted 100 times with the blocking buffer and allowed to react at room temperature for 1 hour. After the reaction, it was washed 3 times with PBST (PBS containing 0.05% Tween 20) at room temperature for 5 minutes and allowed to react at room temperature for 1 hour with an anti-rabbit IgG-HRP-labeled antibody which had been diluted 1,500 times with an HRP-labeled substance dilution buffer (PBS containing 0.1% gelatin). After the reaction, it was washed 3 times with PBST at room temperature for 5 minutes, and then bands were detected by using an luminescence substrate ECL (Amersham). The samples used in lanes 1, 2 and 3 are nmMHCA-introduced cells, and the lane 1 is a solubilized supernatant, lane 2 is an immunoprecipitation product by GAH antibody, and lane 3 is an immunoprecipitation product by human Igs. Also, the samples used in lanes 4, 5 and 6 are mock cells, and the lane 4 is a solubilized supernatant, lane 5 is an immunoprecipitation product by GAH antibody, and lane 6 is an immunoprecipitation product by human Igs.

A band of about 200 Kd was found in the lanes 1 and 2.

It was found from this result that nmMHCA is GAH antibody-specifically immunoprecipitated only in the nmMHCA transient expression cell line.

Confirmation for Preparation of nmMHCA Stable Expression Cell Line by Western Blotting

Each of the stable expression cells after drug selection and mock cells of COS-7 and HCT-15 were recovered by using a scraper, and the SDS-PAGE sample buffer was added thereto, followed by ultrasonic treatment to disrupt the cells. Protein concentrations of the thus prepared samples were determined by using the BCA Protein Assay Kit, and they were subjected to SDS-PAGE (gel concentration 6%), Western blotting and detection by anti-nmMHC antibody.

While nmMHCA was equal to or lower than the detection limit in mock cell of COS-7, a band of about 200 kd corresponding to the molecular weight of nmMHCA was found in the nmMHCA-stably introduced cell line FL11. Also, in the case of HCT-15, while nmMHCA was equal to or lower than the detection limit in mock cell, nmMHCA was detected in the nmMHCA-stably expressing cell lines FL1 and FL2, and it was shown that the expressed amount was large in comparison with FL1.

Immuno-Staining of Nude Mouse Transplantation Cancer Cell Sections Using nmMHCA-Expressing Cell Lines

Various nmMHCA-stably expressing cell lines of HCT-15 were suspended in MatriGel (Becton Dickinson) and subcutaneously transplanted into 2 spots under the back of a nude mouse at a dose of 5×10⁶ cells/spot. Tumor was extracted after confirming its development, and a part thereof was soaked in 10% formalin-PBS to prepare paraffin sections, followed by hematoxylin-eosin (HE) staining at Nara Pathology Institute.

Each section was treated with xylene and ethanol to remove paraffin, soaked in 10 mM sodium citrate, pH 6.0 buffer, exposed to microwave (3 times, 600 W 5 minutes for each), air-cooled by allowing to stand at room temperature for 30 minutes, and then soaked in 5% (w/v) BSA-PBSAz solution for 1 hour. The section was incubated with 66 μg/ml of biotin-labeled F(ab′)₂ fragment of GAH antibody, or 100 μg/ml of biotin-labeled anti-nmMHC antibody, at 37° C. for 2 hours, and then allowed to react with 2.5 times diluted streptoavidin PerCP (Becton Dickinson) under ice-cooling and shade. After the reaction, red fluorescence of PerCP in the same visual field of each section was observed using Olympus incident-light fluorescence microscope BX-50.

The results are shown in FIG. 2. In comparison with the tissue section of mock cell (Mock) used as the control, GAH antibody showed strong red fluorescence for the tissue sections derived from the nmMHCA-stably expressing cell lines (FL1, FL2, FL7). Also, regarding anti-nmMHC antibody, distinct red fluorescence was observed only in the tissue section derived from the nmMHCA-stably expressing cell lines. In addition, the red fluorescence image originated from anti-nmMHC antibody was similar to the red fluorescence image originated from GAH antibody.

It was shown from this result that reactivity of GAH in tissue sections is increased by the introduction of nmMHCA.

Reactivity of GAH Antibody with Intact Cells Using nmMHCA-Stably Expressing Cell Line

An nmMHCA-stably expressing cell line was suspended in MatriGel and subcutaneously transplanted into 2 spots under the back of a nude mouse at a dose of 5×10⁶ cells/spot. Tumor was extracted after confirming its development and cut into thin pieces to collect tumor cells. FITC-labeled GAH antibody or FITC-labeled human immunoglobulin was diluted with human serum (Scanty Bodies Laboratory) to a concentration of 33 μg/ml. Also, FITC-labeled anti-nmMHC antibody or FITC-labeled rabbit IgG was diluted with human serum to a concentration of 50 μg/ml. These antibody solutions were thoroughly mixed with each of tumor cells and allowed to react for 1 hour under shade and ice-cooling. After completion of the reaction, the cells were washed with PBS containing 0.1% sodium azide, and their flow cytometry analysis was carried out in the following manner by using Becton Dickinson BD-LSR. Cells were suspended in FACSflow (buffer attached to the flow cytometer) solution containing 5 μg/ml PI, PI-positive cells, namely dead cells, were excluded from the subject of analysis by gating operation in the same manner as in Example 1, and the mean value of FITC fluorescence intensity per one cell in the intact cell population was calculated. A calibration curve was prepared from the mean value, measured under the same condition, of fluorescence intensity of standard fluorescent beads (Flow Cytometry Standards) wherein the number of bound FITC molecules is already known, and the mean value of each sample was converted into the amount of bound FITC. The thus obtained value was further divided by the F/P value of each FITC-labeled antibody and used as the antibody binding number.

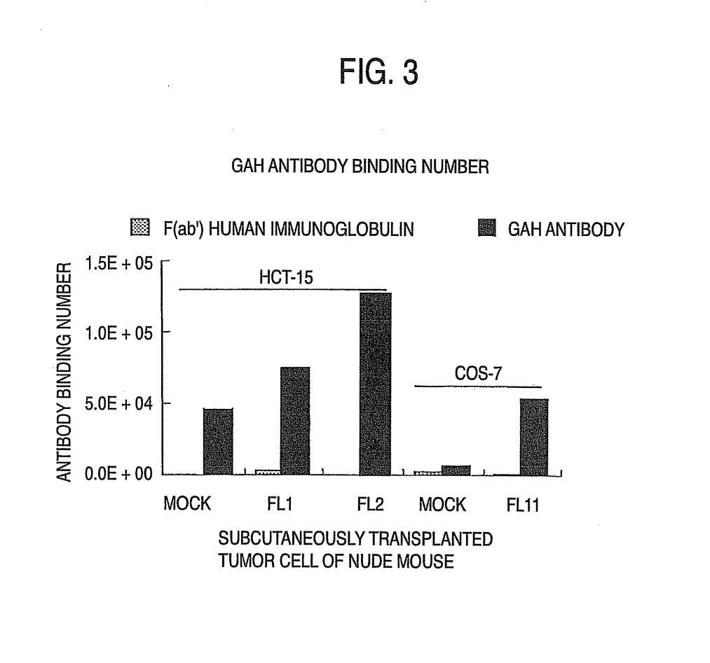

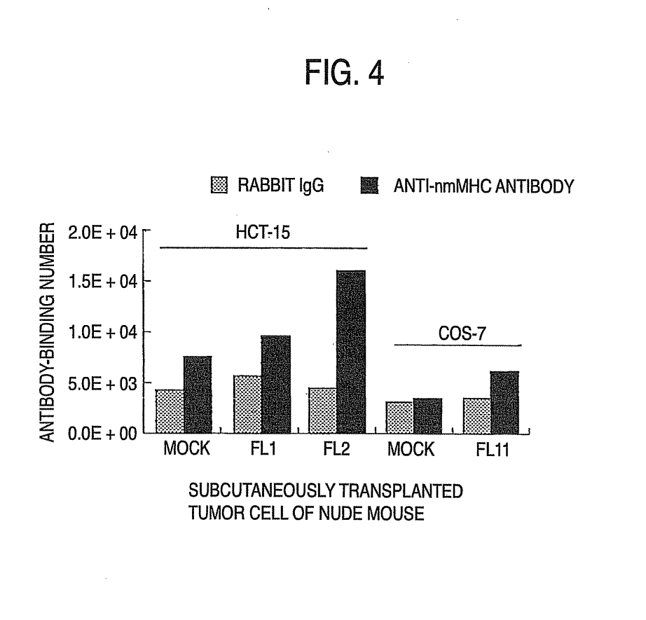

The results are shown in FIG. 3 and FIG. 4. It was shown that the GAH reactivity is increased in the nmMHCA-stably expressing cell lines of both COS-7 and HCT-15, in comparison with the mock cell lines.

It was shown from this result that reactivity of GAH upon the surface of transplanted tumor cells is increased by introducing nmMHCA.

### Example 3

### Confirmation of Cell Surface Reactivity by Anti-nmMHCA Peptide Antibody

The peptides of SEQ ID NOs:20, 21 and 22 in the Sequence Listing as partial peptide sequences of nmMHCA were synthesized and linked to Keyhole Limpet Haemocyanin (KLH), and rabbits were immunized therewith to prepare polyclonal antibodies for respective peptides. Each of the antibodies was purified by using an affinity column in which the corresponding peptide was immobilized on CNBr Sepharose (Amersham Bioscience). Each of the purified polyclonal antibodies and normal rabbit immunoglobulin (normal rabbit IgG: Biogenesis) for control were labeled with FITC, and each antibody was prepared into 50 μg/ml and allowed to react, at 4° C. for 1 hour, with cultured cells of MKN45 and tumor cells formed by subcutaneous transplantation of MKN45. After washing once with PBS, analysis was carried out in PBS containing PI using a flow cytometer (LSR: Becton Dickinson). PI-positive cells, namely dead cells, were excluded from the subject of analysis by gating operation. By calculating the mean channel number (mean value) of FL1 as an index of FITC fluorescence intensity, comparison of respective antibodies was carried out.

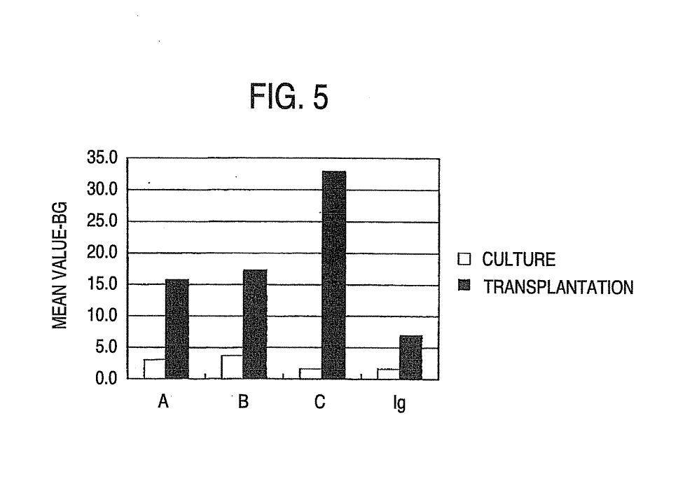

The results are shown in FIG. 5. A, B and C indicate respective anti-nmMHCA peptide antibodies, and Ig indicates normal rabbit immunoglobulin for control. The ordinate shows a value obtained by subtracting a background value (BG) in the case of cells alone from the mean channel number (mean value). In the explanatory notes in the drawing, “culture” indicates MKN45 cultured cells, and “transplantation” indicates tumor cells formed by the transplantation of MKN45.

When the increasing degree of reaction by each antibody in the tumor cells formed by the transplantation of MKN45, in comparison with the MKN45 cultured cells, was calculated by subtracting the reaction of normal rabbit immunoglobulin for control as a background value, 3 times or more of increase was found by the antibody A and antibody B, and 10 times or more of increase by the antibody C.

Based on this result, it was found that, among nmMHCA peptides, the partial sequences represented by SEQ ID NOs:20, 21 and 22 in the Sequence Listing are locally present on the cell surface. In addition, it was found that the existing amounts of the partial sequences represented by SEQ ID NOs:20, 21 and 22 in the Sequence Listing are increased in the tumor cells formed by the transplantation of MKN45 in comparison with the MKN45 cultured cells.

### Example 4

### Confirmation of Antigen Amount and Antitumor Activity

### Cancer Cell Cultured Cell Lines

The human colon cancer cell lines Caco-2, DLD-1 and SW620 were obtained from American Type Culture Collection. The human colon cancer cell line WiDr-Tc and the human esophageal cancer cell line TE-8 were obtained from Cell Resource Center for Biomedical Research, Institute of Development, Aging and Cancer, Tohoku University. The human gastric cancer cell lines HSC-3, MKN-1 and MKN45 and the human colon cancer cell line SW837 were obtained from Immuno-Biological Laboratories. The B37 cell line was established from a human gastric cancer by a conventionally known method.

### Measurement of Antigen Amount on the Surface of Cancer Cells

Subcutaneous tumor transplantation-derived cancer cells of various cancer cell lines were prepared in accordance with the method described in Example 1. Reaction quantities for various cancer cells were detected by flow cytometry in accordance with the method of Example 1, except that F(ab′)₂ fragment of GAH antibody was labeled with FITC, and the reaction was carried out on ice for 1 hour by adjusting concentration of the F(ab′)₂ fragment of GAH antibody to 50 μg/ml. In addition, in order to express the fluorescence detection quantity as antibody reaction quantity (antigen amount) per cancer cell, a fluorescence latex (Flow Cytometry Standard) having known FITC content was used as the standard in the determination.

### Preparation of Immuno-Liposome and Liposome

Using the F(ab′)₂ fragment of GAH antibody, an antibody-linked liposome (immuno-liposome) was prepared in accordance with the method of EP-A-1174126. That is, a doxorubicin (DXR)-enclosed immuno-liposome was formed using a lipid mixture consisting of dipalmitoylphpsphatidylcholine/cholesterol/c-maleimidocaproyldipalmitoylphpsphatidylethenolamine (18/10/0.5 in molar ratio), a polyethylene glycol derivative (PEG) having two polyethylene glycol chains described in the same publication and the F(ab′)₂ fragment of GAH antibody. Particle diameter of the thus obtained immuno-liposome was from 125 nm to 160 nm, and the quantity ratio of F(ab′)₂ fragment of GAH antibody/PEG/DXR/lipid was 0.2:0.8:1:10 (weight ratio).

### Correlation Between Antigen Amount and In Vivo Antitumor Activity

Each of various cancer cells was subcutaneously transplanted into nude mouse to effect formation of a tumor mass. The tumor mass was cut into small pieces of several mm³ and transplanted under the skin membrane of the kidney of another nude mouse [Bennette et al., 1985, Cancer Res., 45, 4963-4969 (1985)]. Starting on the next day, DXR and immuno-liposome were administered from caudal vein (3 mg/kg as DXR) every other week, 3 times in total. The animal was dissected on the 22nd day, and weight of the extracted tumor was measured. A physiological saline-administered group was used as the negative control. As an index of the effect, tumor growth inhibition ratio was calculated based on the following formula.

Tumor growth inhibition ratio (%)=(1−mean tumor weight of drug-treated group/mean tumor weight of negative control group)×100

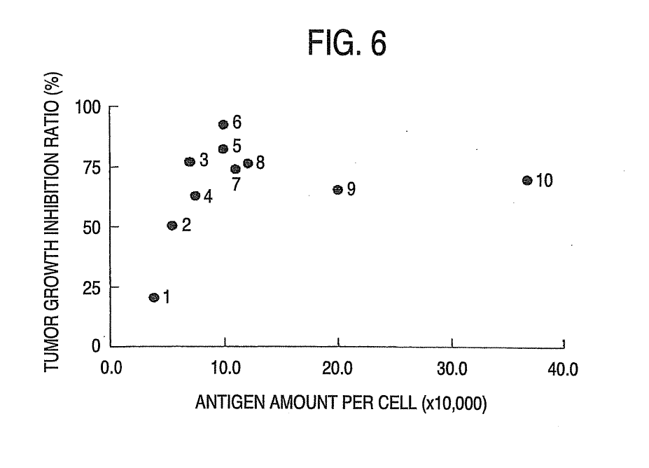

The results are shown in FIG. 6. The ordinate shows the tumor growth inhibition ratio (%), and the abscissa shows the antigen amount per cancer cell. In the drawing, reference numerals represent the following cancer cell lines. 1: Caco-2; 2: DLD-1; 3: HSC-3; 4: SW620; 5: SW837; 6: MKN-1; 7: B-37; 8: MKN45; 9: TE-8; 10: WiDr-Tc.

As a result, it was shown that the tumor growth inhibition ratio was improved as the antigen amount increases on cancer cells having an antigen amount per cancer cell of approximately up to 10⁵/cell, and the antitumor effect reached almost plateau on cancer cells showing an antigen amount of more than that.

### Example 5

### Binding Activity Test on F(ab')₂ Fragment of GAH Antibody

### Preparation of MKN45-Immobilized Plate

MKN45 cell line was inoculated into a flask containing a culture medium and cultured in a CO₂ incubator. When plate face of the flask became a state of being filled with the cells, the culture medium was discarded, and the cells were peeled off by adding a trypsin solution (2.5 g of trypsin 1:250 (Difco) 0.2 g of Na₂EDTA (Sigma) were dissolved with PBS and adjusted to 1,000 ml) and transferred into a centrifugal tube. After centrifugation, the supernatant was removed and the cells were suspended in fresh culture medium. After counting the number of cells, they were suspended in the culture medium to a density of about 4×10⁵ cells/ml, added at 100 μl to each well of 96 well plate and then cultured for 2 days. The culture medium in wells was discarded, PBS was added at 200 μl to each well, and the liquid was discarded. Next, a p-formaldehyde solution was added at 150 μl to each well and allowed to stand still at room temperature for 1 hour, and then the p-formaldehyde solution in the wells was discarded. An operation in which a PBS solution (Sigma) was added bit by bit in each well and then the liquid was discarded was carried out 5 times. The PBS solution (Sigma) was added at 200 μl to each well, and the plate was stored at 4° C.

### Preparation of Standard Solutions and Sample Solutions

Standard solutions 1 to 6 were prepared by adding a dilution solution obtained by diluting 1 g of bovine serum albumin (Sigma) with PBS to a standard stock solution containing 1×10⁶ units/ml of the F(ab′)₂ fragment of GAH antibody. Also, sample solutions 1 to 6 having protein concentrations equivalent to the standard solutions were prepared using the above dilution solution in accordance with the protein content of each sample solution.

| | Concen- | | Concen- |
|---|---|---|---|
| | tration | | tration |
| Standard solutions | (unit/ml) | Sample solutions | (ng/ml) |
| Standard solution 1 | 1000 | Sample solution 1 | 1000 |
| Standard solution 2 | 500 | Sample solution 2 | 500 |
| Standard solution 3 | 250 | Sample solution 3 | 250 |
| Standard solution 4 | 125 | Sample solution 4 | 125 |
| Standard solution 5 | 62.5 | Sample solution 5 | 62.5 |
| Standard solution 6 | 31.25 | Sample solution 6 | 31.25 |

### Operation Method

An operation in which the liquid in wells of the plate was discarded, 200 μl of PBS is added to each well and then the liquid was discarded was carried out 5 times. Each of the standard solutions and sample solutions was added at 50 μl/well and allowed to stand still at 37° C. for 2 hours, and then the liquid discarded. An operation in which 200 μl of PBS was added to each well and then the liquid was discarded was carried out 5 times.

Next, 50 μl of a horseradish peroxidase-labeled goat anti-human κ chain antibody solution (Cappel) was added to each well and allowed to stand still at 37° C. for 1 hour. The liquid was discarded, 200 μl of a washing liquid was added to each well and allowed to stand still at 37° C. for 5 to 10 minutes, and then the liquid was discarded. Subsequently, an operation in which 200 μl of the washing liquid was added to each well and then the liquid was discarded was carried out 5 times.

To each well, 100 μl of an o-phenylenediamine solution (Sigma) was added and allowed to stand still at room temperature for 1 to 2 minutes under shade. To each well, 100 μl of a stop solution was added and mixed by gentle shaking.

Using a microplate reader, absorbances A1 and A2 at wavelengths of 490 nm and 650 nm in each well were measured to calculate (A1-A2).

A calibration was obtained by plotting the concentration of standard solutions as the abscissa and the absorbance (A1-A2) as the ordinate. From the absorbance of each sample solution, titer per 1 ml of each sample solution (unit/ml) was obtained, and titer per 1 mg of the sample (unit/mg) was calculated.

As a result, it was found that the use of the F(ab′)₂ fragment of GAH antibody having a binding activity titer of from 0.8 to 1.2×10⁶ unit/mg is useful.

### INDUSTRIAL APPLICABILITY

According to the present invention, an antigen having a part which is exposed on the surface of a cell at the formation of a tumor mass can be provided.

Also, the present application was filed by claiming the priority of Japanese application No. 2002-291953, the entire contents of which are incorporated hereinto by reference.

1. A method of treating cancer, which comprises administering an antibody against a non-muscular myosin heavy chain type A or a mutant thereof, or a F(ab′)₂ fragment thereof to a patient in need thereof,
provided that the antibody is not an antibody wherein the heavy chain hypervariable region comprises SEQ ID NOs: 1, 2, and 3, and the light chain hypervariable region comprises SEQ ID NOs: 4, 5, and 6, nor a variant antibody thereof, which is obtainable by making an insertion, deletion, substitution, and/or addition of one or more amino acid residues to the amino acid sequences of the antibody with the limitation that such modification does not adversely affect the reactivity of the antibody against the antigen, thereby treating cancer in the patient.

2. The method according to claim 1, wherein the cancer is gastric cancer, breast cancer, colon cancer, or esophageal cancer.

3. The method according to claim 1, wherein the binding activity of the antibody or F(ab′)₂ fragment thereof is from 0.5×10⁶ units/mg to 2.0×10⁶ units/mg.

4. The method according to claim 2, wherein the binding activity of the antibody or F(ab′)₂ fragment thereof is from 0.5×10⁶ units/mg to 2.0×10⁶ units/mg.

5. The method according to claim 1, wherein the binding activity of the antibody or F(ab′)₂ fragment thereof is from 0.7×10⁶ units/mg to 1.5×10⁶ units/mg.

6. The method according to claim 2, wherein the binding activity of the antibody or F(ab′)₂ fragment thereof is from 0.7×10⁶ units/mg to 1.5×10⁶ units/mg.

7. The method according to claim 1, wherein the binding activity of the antibody or F(ab′)₂ fragment thereof is from 0.8×10⁶ units/mg to 1.2×10⁶ units/mg.

8. The method according to claim 2, wherein the binding activity of the antibody or F(ab′)₂ fragment thereof is from 0.8×10⁶ units/mg to 1.2×10⁶ units/mg.

9. An antibody against a non-muscular myosin heavy chain type A or a mutant thereof, or a F(ab′)₂ fragment thereof for use as a cancer targeting agent,
wherein the antibody or F(ab′)₂ fragment thereof is linked to a liposome in which an anti-cancer agent and/or a labeling agent is contained,
provided that the antibody is not an antibody wherein the heavy chain hypervariable region comprises SEQ ID NOs: 1, 2, and 3, and the light chain hypervariable region comprises SEQ ID NOs: 4, 5, and 6, nor an variant antibody thereof, which is obtainable by making an insertion, deletion, substitution, and/or addition of one or more amino acid residues to the amino acid sequences of the antibody with the limitation that such modification does not adversely affect the reactivity of the antibody against the antigen.

10. The antibody or an F(ab′)₂ fragment thereof according to claim 9, wherein the targeted cancer is gastric cancer, breast cancer, colon cancer, or esophageal cancer.

11. The antibody or an F(ab′)₂ fragment thereof according to claim 9, wherein the binding activity of the antibody or an F(ab′)₂ fragment thereof is from 0.5×10⁶ units/mg to 2.0×10⁶ units/mg.

12. The antibody or an F(ab′)₂ fragment thereof according to claim 9, wherein the binding activity of the antibody or an F(ab′)₂ fragment thereof is from 0.7×10⁶ units/mg to 1.5×10⁶ units/mg.

13. The antibody or an F(ab′)₂ fragment thereof according to claim 9, wherein the binding activity of the antibody or an F(ab′)₂ fragment thereof is from 0.8×10⁶ units/mg to 1.2×10⁶ units/mg.

14. The antibody or an F(ab′)₂ fragment thereof according to claim 9, wherein the liposome contains an anti-cancer agent.

15. The antibody or an F(ab′)₂ fragment thereof according to claim 11, wherein the liposome contains an anti-cancer agent.

16. The antibody or an F(ab′)₂ fragment thereof according to claim 12, wherein the liposome contains an anti-cancer agent.

17. The antibody or an F(ab′)₂ fragment thereof according to claim 13, wherein the liposome contains an anti-cancer agent.

18. A method of treating cancer, which comprises administering an antibody according claim 9 to a patient in need thereof, thereby treating cancer in the patient.

19. A method of treating cancer, which comprises administering an antibody according claim 10 to a patient in need thereof, thereby treating cancer in the patient.

20. A method of treating cancer, which comprises administering an antibody according claim 11 to a patient in need thereof, thereby treating cancer in the patient.

21. A method of treating cancer, which comprises administering an antibody according claim 12 to a patient in need thereof, thereby treating cancer in the patient.

22. A method of treating cancer, which comprises administering an antibody according claim 13 to a patient in need thereof, thereby treating cancer in the patient.

23. A method of treating cancer, which comprises administering an antibody according claim 14 to a patient in need thereof, thereby treating cancer in the patient.

24. A method of treating cancer, which comprises administering an antibody according claim 15 to a patient in need thereof, thereby treating cancer in the patient.

25. A method of treating cancer, which comprises administering an antibody according claim 16 to a patient in need thereof, thereby treating cancer in the patient.

26. A method of treating cancer, which comprises administering an antibody according claim 17 to a patient in need thereof, thereby treating cancer in the patient.
